(19) United States
(12) Patent Application Publication
(10) Pub. No.: US 2008/0318243 A1
(43) Pub. Date: Dec. 25, 2008

(54) DNA measuring system and method

(75) Inventors: Takanobu Haga, Kokubunji (JP); Masao Kamahori, Kokubunji (JP); Tomoyuki Sakai, Kokubunji (JP); Yu Ishige, Tokyo (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 12/213,376

(22) Filed: Jun. 18, 2008

(30) Foreign Application Priority Data
Jun. 21, 2007 (JP) 2007-164231

(51) Int. Cl. C12Q 1/68

(52) U.S. Cl. 435/6

(57) ABSTRACT

The present invention provides a DNA sequencer using a FET sensor, capable of long-base decoding. Target DNAs are immobilized on the surfaces of spherical fine particles, the fine particles are disposed in the vicinity of metal electrodes each of which is connected electrically to a corresponding one of conductive wirings of the FET sensor and partly has a spherical surface capable of contacting with the fine particles, and the FET sensor detects a change in interfacial potential incident to an extension reaction of DNA molecules containing a hybridization of the target DNA and probe DNA.

### CLAIM OF PRIORITY

The present application claims priority from Japanese application JP 2007-164231 filed on Jun. 21, 2007, the content of which is hereby incorporated by reference into this application.

### BACKGROUND OF THE INVENTION

1. Field of the Invention

The present invention relates to a measuring system for making a measurement on a biological substance, such as DNA (deoxyribonucleic acid) or RNA (ribonucleic acid), as unmodified, and a measuring method using the same, and more particularly to a measuring system and method using a field effect transistor (FET).

2. Description of the Related Art

Recent marked advances in nucleotide sequence analysis technology have led to determination of a reference sequence for a whole human genome, thus enabling a comparison for determining directly the dissimilarity in genes between individuals. As for a disease-related gene in particular, gene analysis using SNPs is used to narrow down a potential region as a candidate, thus enabling a comparison of sequences in the region between a healthy individual and a patient. However, the use of one existing DNA sequencer for genome analysis for one person requires an enormous cost and a long time, which in turn creates a need for a DNA sequencer capable of achieving far lower cost and higher throughput. Against such a background, the National Institutes of Health of the U.S. are pursuing the development of DNA analysis technology, setting their goal of achieving genome decoding for one person at reasonable cost. In order to realize the DNA sequencer capable of achieving far lower cost and higher throughput than the conventional DNA sequencer, there is development of a massively parallel DNA sequencer designed to increase the number of reads concurrently processed by one digit or more. To increase the number of reads concurrently processed, the massively parallel DNA sequencer has a micro-miniaturized, high-density reactor for sequencing, thereby making it possible to reduce a dose of reagent for use and thus to lower the cost of decoding.

The currently-developed massively parallel DNA sequencers include a pyrosequencing apparatus for implementing, at high density, a pyrosequencing method that involves hybridizing target DNAs with probe DNAs; changing a pyrophosphoric acid formed by a polymerase extension reaction into ATP (adenosine triphosphate); causing luciferin to react with the ATP to thereby produce bioluminescence; and detecting this bioluminescence to thereby determine a substrate (namely, deoxyribonucleotide triphosphate (dNTP)) captured by the polymerase extension reaction, thereby sequentially determining nucleotide sequence (see Nature 2005, Vol. 437, pp. 376-380), and a single molecule DNA sequencing apparatus for implementing, at high density, a fluorescence detection method that involves hybridizing target DNAs with probe DNAs immobilized on a glass surface; and detecting a fluorescently-labeled dNTP captured by a polymerase extension reaction to thereby determine the dNTP captured by the polymerase extension reaction, whereby sequentially determining nucleotide sequence (see PNAS 2003, Vol. 100, pp. 3960-3964).

For a micro-miniaturized, high-density reactor for pyrosequencing, the above-mentioned pyrosequencing apparatus uses emulsion (em) PCR amplification to place beads of about 30 μm in diameter, having amplified target DNA fragments immobilized thereon, one by one, in wells of about 45 μm in diameter, arranged in an array. In the pyrosequencing, the array of the wells is placed in a flow cell having an injection port and an ejection port, and four types of dNTP solutions are injected into a flow cell through the port one after another. According to the principle of the pyrosequencing, luminescence produced incident to an extension reaction undergoes imaging on a CCD (charge coupled device) through optical fibers corresponding to the wells, and an average of about 100 bases for target DNA molecules immobilized on the beads are sequenced. On that occasion, the beads have different target DNAs immobilized thereon, respectively, and thus, 450,000 types of target DNAs can be processed in parallel by a single run.

The above-mentioned single molecule DNA sequencing apparatus uses two types of fluorophore (e.g., the Cy3 and the Cy5) for the labeling of the probe DNA and the dNTP that acts as the substrate, respectively, and uses two types of lasers (e.g., with wavelengths of 532 nm and 635 nm, respectively) for detection of the labeled probe DNA and substrate. A single target DNA molecule is immobilized on the glass surface by utilizing a biotin-avidin bond, and then the Cy3-labeled probe DNA is hybridized with the target DNA molecule. At this time, the Cy3 is fluorescently detected by evanescent irradiation with the laser with a wavelength of 532 nm to thereby find the location of the target DNA molecule. Then, the introduction of polymerase and the Cy5-labeled dNTP with a type of base (where N denotes any one of A, C, G and T) into the solution leads to the capture of the fluorescence-labeled dNTP molecules into an extension strand of the probe DNA, only when a complementary extension reaction occurs. The presence or absence of the extension reaction is determined by detection of fluorescence produced by evanescent irradiation with the laser with a wavelength of 635 nm. After that, the Cy5 photobleaches by irradiation with the high-power laser with a wavelength of 635 nm. Nucleotide sequence determination for the target DNA molecules can be accomplished by sequentially repeating the above extension reaction process for the dNTP. This method enables parallel processing of 200 to 300 target DNAs in a field of view of 100 μm in diameter, and thus enables parallel processing of 12,000,000 target DNAs in a region 25 by 25 millimeters square, using an automated scan.

On the other hand, there has been a report on a method that involves immobilizing probe DNA on a gate insulating layer formed on a FET sensor across its source and drain; and detecting a change in interfacial potential on the insulating layer incident to an extension reaction of the probe DNA hybridized with the target DNA, directly through a change in current value across the source and drain, thereby effecting sequence determination for DNA, without having to use the reagent and enzyme for bioluminescence reaction or the fluorophore for fluorescence detection as mentioned above (see Angewandte Chemie 2006, Vol. 45, pp. 2225 to 2228).

The above-mentioned FET sensor method uses a deposit of a SiO₂ (silicon dioxide) layer and Si₃N₄ (silicon nitride) thereon which acts as an overcoat film, as the gate insulating layer formed on the FET sensor across its source and drain The probe DNA is immobilized on the surface (namely, the Si₃N₄ surface) of the FET sensor by silane coupling and is hybridized with the target DNA. After that, a solution containing DNA polymerase and dNTP with a type of base (where N refers to any one of A, C, G and T) is introduced to induce the extension reaction. A dNTP molecule has one phosphoric group, and is negatively charged in an aqueous solution. Thus, the capture of the dNTP molecules into an extension strand of probe DNA molecules causes a change in charge density on the surface of the FET sensor and hence the interfacial potential changes. The change in the interfacial potential can be detected through the change in the current value across the source and drain. Thus, the amount of capture of the dNTP can be measured, based on the amount of change in the current value across the source and drain. Nucleotide sequence determination for target DNA molecules can be accomplished by repeating the above capture reaction process for the dNTP in a stepwise fashion, while stepping the type of base, for example in turn from A (adenine) to C (cytosine), G (guanine), and T (thymine). Sequencing technology using the FET sensor, as described in the above document, can lower the cost of decoding, because it does not use an expensive reagent for luminescence or fluorescence. Also, a typical semiconductor fabrication process may be used to form FET sensors in an array at high density. With setting points of detection at high density, photo-detection such as luminescence measurement or fluorescence measurement presents the problem of crosstalk, whereas the FET sensor presents no problem of crosstalk because of using potential measurement as its basic principle.

### SUMMARY OF THE INVENTION

The above-mentioned FET-based DNA sequencer immobilizes the probe DNA on the gate insulating layer formed on the FET sensor across its source and drain, hybridizes the target DNA with the probe DNA, and detects the extension reaction and the change in the interfacial potential on the insulating layer incident to the extension reaction. Thus, the DNA sequencer has the problem of a short length of base detectable in principle, and further has the problem of having difficulty in the reuse of the FET sensor.

The detectable number of bases (or equivalently, the readable number of bases) is determined by the relationship between a change in electric charge incident to the extension reaction of the probe DNA hybridized with the target DNA and the change in the interfacial potential involved in the change in the electric charge. The influence of the electric charge on the surface of the FET sensor (e.g., the Si₃N₄ surface that forms a part of the gate insulating film, as employed in Angewandte Chemie 2006, Vol. 45, pp. 2225-2228) upon the interfacial potential decreases with increasing distance from the surface of the sensor to an extension reaction region. Thus, as the extension reaction region becomes farther away from the surface of the sensor with the proceeding of the extension reaction of the probe DNA hybridized with the target DNA, the amount of change in the interfacial potential per extension reaction of base becomes smaller, so that it becomes difficult to detect the extension reaction. Generally, the limit of the distance from the surface of the sensor at which the interfacial potential is detectable is determined by Debye length obtained mainly by ionic species in the solution and their ionic strengths, and the like. Under the condition of a low concentration of buffer (e.g., 2.5 mM) specialized for increasing the Debye length as employed in Angewandte Chemie 2006, Vol. 45, pp. 2225-2228, the Debye length was about 10 nm. Thus, the limit of the detectable number of bases is 30 in theory, allowing for the size of base (e.g., 0.34 nm). Actually, the readable number of bases was approximately 10. Also, the limit of the readable number of bases is 20 in practice, allowing for the length of a portion hybridized with the probe DNA or the length of a linker bonded to the probe DNA for immobilization on the surface of the sensor, and 50 bases or more are required to uniquely map sequence information onto the reference genome sequence after sequence determination (see Nucleic Acids Research 2005, Vol. 33. e171), which in turn makes it difficult to use this DNA sequencer for typical sequencing.

As for the reuse of the FET sensor, it is necessary to remove the DNA molecules after use for sequencing, if the probe DNA or the target DNA is immobilized directly on the surface of the sensor. However, this requires a complicated process using a special chemical solvent or the like, thus makes it difficult to reuse the FET sensor, and hence leads to an increase in the running cost.

In order to solve the above problems, according to the present invention, a spherical fine particle having immobilized thereon double-stranded DNA containing a hybridization of a probe DNA and a target DNA is disposed on a metal electrode surface of an extended gate FET sensor that is a metal electrode having a spherical surface on which a sensing unit is in contact with the fine particle, and an extension reaction of the double-stranded DNA is detected through a change in interfacial potential on the surface of the sensor. As employed here, the extended gate FET sensor is an insulated gate field effect transistor sensor in which the metal electrode that is the sensing unit is connected to a gate of an insulated gate field effect transistor by a conductive wiring.

Generally, the field effect transistor involves leakage current, and thus, a drain current value varies with measuring time independently of the change in the interfacial potential on the insulating layer. For this reason, for detection of the extension reaction through the change in the interfacial potential on the surface of the sensor, it is desirable that a change in the drain current value caused by the change in the interfacial potential after the extension reaction be greater than that caused by the leakage current. As the above condition, the shape of the sensing unit of the FET sensor, as shown in FIG. 1, is determined so that Equations (1) and (2) can be satisfied:

r₂−\sqrt{r₂²−2(1−cos θ)r₁r₂+2(1−cos θ)r₁²}<D (1)

r₁<r₂ (2)

where r₁ denotes the radius of a fine particle 101; r₂, the curvature radius of the spherical surface on the surface of a metal electrode 103; D, the Debye length; θ, a parameter that takes on a value that satisfies Equation (3); Iᵣ, the leakage current value of the FET sensor; tₐ, the time interval between interfacial potential measurements; C, a coefficient of conversion from coulomb to electron number; and d, the density of the double-stranded DNA immobilized on the fine particle.

2π(1−cos θ)r₁²d>IᵣtₐC (3)

In order to suppress deterioration in the reproducibility of measurement caused by a change in the position of the fine particle due to Brownian motion thereof, there is provided a mechanism for pressing the fine particle against the surface of the metal electrode, using a flow by a pressure wave, an attraction by a magnetic field, a flow by a change in temperature, or the like. Also, there is provided a mechanism for separating the fine particle from the surface of the metal electrode at the time of a solution change, using the flow by the pressure wave, the attraction by the magnetic field, the flow by the change in temperature, or the like. One and the same mechanism may be used as the mechanism for pressing the fine particle against the surface of the metal electrode and that for separating the fine particle from the surface of the metal electrode.

The present invention enables sequence determination for DNA with a long base without being limited by the Debye length, which becomes a problem with the existing FET-based DNA sequencer. The use of the metal electrode having the spherical surface in contact with the fine particle as the sensing unit of the FET sensor enables a contact of the spherical surface of the sensing unit with the surface of the fine particle over a wide range, and thus enables the arrangement of a larger number of target DNAs within the Debye length, as compared to the use of a flat metal electrode. Also, the inside of the metal electrode is of equal potential, and thus, the change in the interfacial potential that occurs in any region on the surface of the sensor can effect an equal change in the potential of the gate insulating film regardless of the shape of the metal electrode, thus enabling high-accuracy detection.

Also, the immobilization of the probe DNA or the target DNA on the spherical fine particle, rather than that directly on the surface of the sensor, enables the reuse of the FET sensor merely by removing the fine particle after sequencing.

Further, the pressing of the fine particle against the surface of the metal electrode using the flow by the pressure wave, the attraction by the magnetic field, the flow by the change in temperature, or the like enables suppressing the deterioration in the reproducibility of measurement caused by a shift in the position of the fine particle. It is sufficient for the fine particle to be pressed against the surface of the metal electrode simultaneously only when potential measurement by the FET sensor is carried out. Also, the separation of the fine particle from the surface of the metal electrode at the time of the solution change enables an effective solution change.

### BRIEF DESCRIPTION OF THE DRAWINGS

FIG. 1 is a schematic view showing the relationship between a spherical surface of a metal electrode and that of a fine particle.

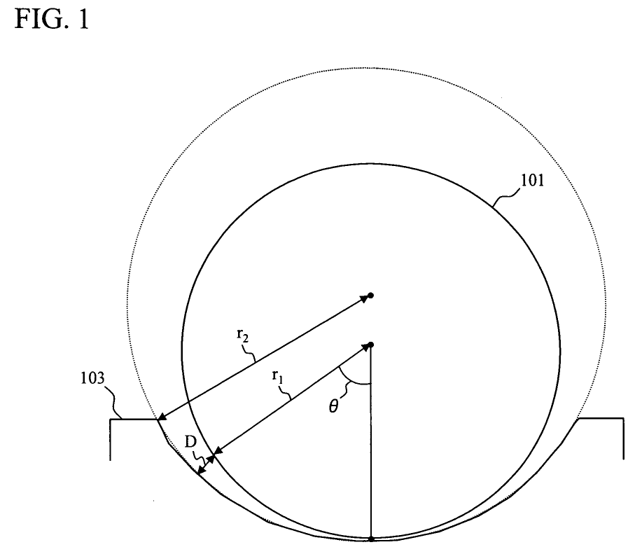

FIG. 2 is a view showing an example of a system configuration of a FET-based DNA sequencer according to the present invention.

FIG. 3 is a flowchart showing processes for DNA sequence analysis.

FIG. 4 is a wiring diagram for measurement of a drain current value from multiple FETs.

FIGS. 5A and 5B are a front view of a FET sensor unit of a fourth embodiment and a cross-sectional view taken along the line B-B of FIG. 5A, respectively.

### DETAILED DESCRIPTION OF THE PREFERRED EMBODIMENTS

Embodiments of the present invention will be described below.

### First Embodiment

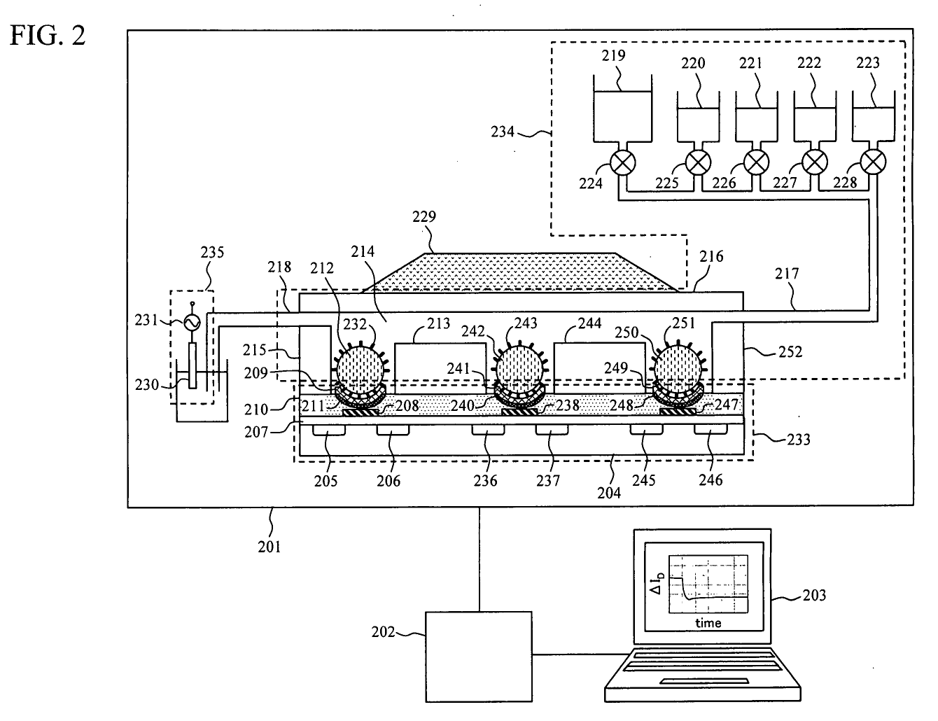

FIG. 2 shows an example of a system configuration of a FET-based DNA sequencer according to the present invention. The system is configured of a measuring unit 201, a signal processing circuit 202, and a data processing device 203. Further, the measuring unit 201 has four structural components including: a FET sensor unit 233, a chamber unit 234, a fine particle control unit 229, and a reference electrode unit 235. Details of these components will be described below.

The FET sensor unit 233 has multiple FETs formed on a silicon substrate 204, includes multiple combinations of: a source 205; a drain 206; a metal electrode 209 formed on an adhesive layer 211 connected to a gate insulating film 207 through a conductive wiring 208; a source 236; a drain 237; a metal electrode 241 formed on an adhesive layer 240 connected to the gate insulating film 207 through a conductive wiring 238; a source 245; a drain 246; and a metal electrode 249 formed on an adhesive layer 248 connected to the gate insulating film 207 through a conductive wiring 247, and is coated with an insulating film 210 except for the metal electrodes.

A fabrication process for the FET sensor unit 233 will be described, dividing the FET sensor unit 233 into two parts: a FET basic configuration section, and the metal electrode having a spherical surface that is a feature of the present invention.

Existing semiconductor fabrication technology was used to form the FET basic configuration section, i.e., the sources 205, 236 and 245, the drains 206, 237 and 246, the gate insulating film 207, and the conductive wirings 208, 238 and 247. Firstly, the silicon substrate 204 was oxidized to form the gate insulating film 207 made of SiO₂. Thereafter, in order to form the conductive wirings 208, 238 and 247 made of polysilicon arranged in an array, the gate insulating film 207 was coated throughout its entire surface with the polysilicon, and then, processes of resist coating, patterning, development, and etching were carried out in this order. The sources and the drains were formed by ion implantation using the conductive wirings 208, 238 and 247 as a mask. Polysilicon is desirable for material of the conductive wirings 208, 238 and 247, since it is highly compatible with a so-called self-alignment process in which ion is implanted through a polysilicon gate to form the source and the drain. As to the gate insulating film 207, silicon oxide (SiO₂), silicon nitride (SiN), aluminum oxide (Al₂O₃), tantalum oxide (Ta₂O₅), and other material may be used alone or in combination for formation. Further, the gate insulating film 207 may also employ a two-layer structure formed by laminating silicon nitride (SiN), aluminum oxide (Al₂O₃), or tantalum oxide (Ta₂O₅) on silicon oxide (SiO₂) in order to keep good performance of the transistor.

Description will now be given below with regard to a fabrication process for the metal electrodes 209, 241 and 249 each having a spherical concave surface fitted to the shape of corresponding one of fine particles 212, 242 and 250. A SiO₂ film was formed as the insulating film 210 on the conductive wiring 208, 238 and 247 by CVD (chemical vapor deposition), and then the insulating film was coated throughout its entire surface with a resist. Then, dot patterning was performed on locations where the metal electrodes 209, 241 and 249 were disposed (i.e., on the conductive wirings 208, 238 and 247), and development was performed there. Each of the patterned parts was subjected to isotropic etching to thereby form a spherical surface on the insulating film 210. After that, tungsten (W) was sputtered as the adhesive layers 211, 240 and 248 for providing electrical connections between the metal electrodes 209, 241 and 249 and the conductive wirings 208, 238 and 247, respectively, and then gold (Au) was sputtered as the metal electrodes 209, 241 and 249. Thereafter, the metal electrodes 209, 241 and 249 each having the spherical surface were formed in an array by resist coating, patterning, development, and etching in the same manner as described above.

In the first embodiment, in order to bond the metal electrodes 209, 241 and 249 and the insulating film 210 firmly, the gold (Au) that forms the metal electrodes 209, 241 and 249 was formed on the SiO₂ film that forms the insulating film 210, with the tungsten (W), which forms the adhesive layers 211, 240 and 248, in between. However, the metal electrodes 209, 241 and 249 may also be formed directly on the conductive wirings 208, 238 and 247 without interposing the adhesive layers. Incidentally, it is desirable for a material for the metal electrodes 209, 241 and 249 to have high chemical stability and stable potential for a direct contact with a sample solution, and to have high affinity for a biological material so as to immobilize the biological material, and noble metal such as gold (Au), platinum (Pt), silver (Ag), or palladium (Pd) can be used.

In the first embodiment, beads having a diameter of 200 μm (or equivalently, a radius r₁ of 100 μm) were used as the fine particles 212, 242 and 250. Extension reaction time, time required for solution change, and the time interval tₐ required for measurement of the interfacial potential were set to 5 minutes, and the leakage current value Iᵣ of the FET sensor was set equal to 1 fA. The curvature radius r₂ required to achieve high sensitivity of the spherical surface of the metal electrode was calculated, from the foregoing Equations (1), (2) and (3), to lie between 100 and 108 μm, both inclusive (100≦r₂≦108 (μm)). Thus, the metal electrodes 209, 241 and 249 were fabricated so that the curvature radius of the spherical surfaces thereof would be equal to 100 μm. However, the range of the curvature radius r₂ is just an index for higher-sensitivity sequence decoding, and the effect of the present invention is not impaired even if the curvature radius r₂ deviates from the above range.

The chamber unit 234 is configured of: the fine particles 212, 242 and 250; sectioning layers 213 and 244; a solution tank 214; spacers 215 and 252; a cover 216; a solution injection port 217; a solution ejection port 218; a cleaning solvent container 219; a dATP solution container 220; a dTTP solution container 221; a dGTP solution container 222; a dCTP solution container 223; a cleaning solvent feed valve 224; a dATP solution feed valve 225; a dTTP solution feed valve 226; a dGTP solution feed valve 227; and a dCTP solution feed valve 228. The sectioning layers 213 and 244 for preventing the fine particles 212, 242 and 250 from moving to an adjacent well were fabricated by resist coating, patterning, development, and etching in the same manner as described above.

The thickness of the sectioning layers 213 and 244 is not particularly limited; however, desirably, the thickness of the sectioning layers 213 and 244 is equal to or more than the radius of the fine particles 212, 242 and 250 in order that the fine particles can be efficiently disposed one by one in the wells when the fine particles 212, 242 and 250 are developed in the solution tank 214. Also, for efficient solution change, it is desirable to minimize the thickness of the sectioning layers 213 and 244 within the above range. In the first embodiment, the radius of the fine particles 212, 242 and 250 was set equal to 100 μm, and thus, the thickness of the sectioning layers 213 and 244 was set equal to 100 μm. Also, polyimide was used as a material for the sectioning layers 213 and 244 in this embodiment, but it is not particularly limited. If the spacers 215 and 252 are too thick, the distance between the sectioning layers 213 and 244 and the cover 216 becomes longer, so that the fine particles 212, 242 and 250 can possibly move to the adjacent wells. In order to prevent this movement, it is required that the spacers be thinner than the thickness of the sectioning layers 213 and 244 plus the diameter of the fine particles 212, 242 and 250.

For efficient solution change, it is desirable to maximize the thickness of the spacers 215 and 252 within the above limits. In the first embodiment, the thickness of the sectioning layers 213 and 244 was set to 100 μm and the diameter of the fine particles 212, 242 and 250 was set to 200 μm, and thus, a polyethylene film of 250 μm was used for the spacers 215 and 252. However, a material therefor is not specified. The cleaning solvent container 219 was filled with a phosphate buffer solution (0.025 M Na₂HPO₄/0.025 M KH₂PO₄, pH 6.86), and the dATP solution container 220, the dTTP solution container 221, the dGTP solution container 222, and the dCTP solution container 223 were filled with KCl (50 mM), Tris-HCl (20 mM, pH 8.4), MgCl₂ (3 mM), Klenow Fragment (0.1 UμL⁻¹), and dNTP (5 mM), respectively (the DATP solution container 220 was filled with a dATP solution; the dTTP solution container 221, with a dTTP solution; the dGTP solution container 222, with a dGTP solution; and the dCTP solution container 223, with a dCTP solution).

The fine particle control unit 229 is configured to extricate the fine particles 212, 242 and 250 from the metal electrodes 209, 241 and 249 at the time of solution change and to settle the surfaces of the fine particles 212, 242 and 250 within the Debye length on the surfaces of the metal electrodes 209, 241 and 249 at the time of interfacial potential detection. In the first embodiment, a pressure wave generator was used as the fine particle control unit 229.

The reference electrode unit 235 consists of a reference electrode 230 and a high-frequency power supply 231. A silver/silver chloride electrode having a saturated potassium chloride solution in an internal solution was used as the reference electrode 230. In the first embodiment, the reference electrode unit 235 was disposed in a container that receives the solution ejected from the solution ejection port 218 as shown in FIG. 2. However, the reference electrode unit 235 may also be disposed in another place, as long as it is in contact with the solution introduced into the solution tank 214. Also, a direct-current power supply may be used in place of the high-frequency power supply.

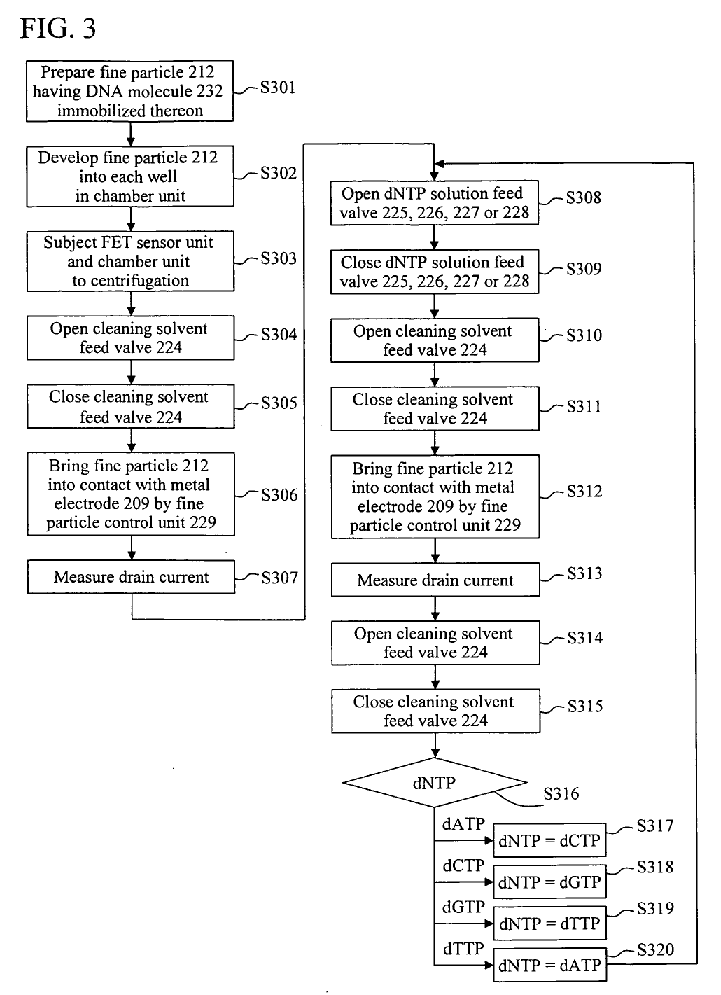

A process for sequence analysis will be described below with reference to FIG. 3. A method for immobilization of probe DNAs 232, 243 and 251 on the fine particles 212, 242 and 250 and a method for hybridization of target DNAs with the immobilized probe DNAs followed the methods described in Nature 2005, Vol. 437, pp. 376-380. Different target DNA fragments were immobilized one by one on the fine particles 212, 242 and 250, respectively, and emPCR amplification was used to obtain the fine particles 212, 242 and 250 each having the corresponding multiple target DNA molecules immobilized on its surface (at step S301). Although a sepharose bead, whose specific gravity is close to 1, was used as a material for the fine particles 212, 242 and 250 for an efficient emPCR amplification process, other materials may also be used.

The solution containing the fine particles 212, 242 and 250 having immobilized thereon the DNA molecules prepared by the above method was developed in the solution tank 214 with the cover 216, the solution injection port 217 and the solution ejection port 218 removed (at step S302). Thereafter, the FET sensor unit 233 and the chamber unit 234 were subjected to centrifugation with the cover 216 attached to dispose the multiple fine particles 212, 242 and 250 one by one in the wells sectioned by the sectioning layers 213 and 244 (at step S303). Incidentally, during the centrifugation, a paraffin film was used to block joints of the removed solution injection port 217 and solution ejection port 218 in order to prevent the solution in the solution tank 214 from leaking through the joints. After that, the solution injection port 217 and the solution ejection port 218 were attached and the cleaning solvent feed valve 224 was opened (at step S304). The phosphate buffer solution (0.025 M Na₂HPO₄/0.025 M KH₂PO₄, pH 6.86) was injected into the solution tank 214 through the solution injection port 217, air bubbles present in the solution tank were removed through the ejection port, and then the valve was closed (at step S305).

For detection of the interfacial potential before the extension reaction, the pressure wave generator installed in the fine particle control unit 229 was used to measure the interfacial potential while continuously emitting pulses of pressure waves at a pressure of about 10 atmospheres toward the metal electrodes 209, 241 and 249 (at step S306). At this time, the fine particles 212, 242 and 250 undergoing Brownian motion in the wells were pressed against the spherical surfaces of the metal electrodes 209, 241 and 249 by the pressure waves, so that the interfacial potential changed along with pressure wave generation. A semiconductor parameter analyzer that is a constituent part of the signal processing circuit 202 was used for interfacial potential measurement. The high-frequency power supply 231 was used to apply a voltage having a frequency of 1 MHz, an amplitude of 0.2 V and a bias of 0.1 V to the reference electrode 230, while 1 V is applied across the sources 205, 236 and 245 and the drains 206, 237 and 246, respectively, a change in current was monitored in real time, and the drain current value was recorded by the signal processing circuit 202 and the data processing device 203 (at step S307). The recorded drain current value was converted into the interfacial potential, based on relationship characteristics between a gate voltage and drain current measured separately. The average of interfacial potential changes detected multiple times in the wells is indicated by V₀; and a standard deviation thereof, by ΔV₀. For interfacial potential measurement, the FET sensor unit and the chamber unit were covered with a blackout curtain and thereby shielded from light in order to reduce the entry of light, which can possibly cause noise, into the conductive wiring and the gate and thereby to achieve a high S/N ratio. However, light shield operation is not essential.

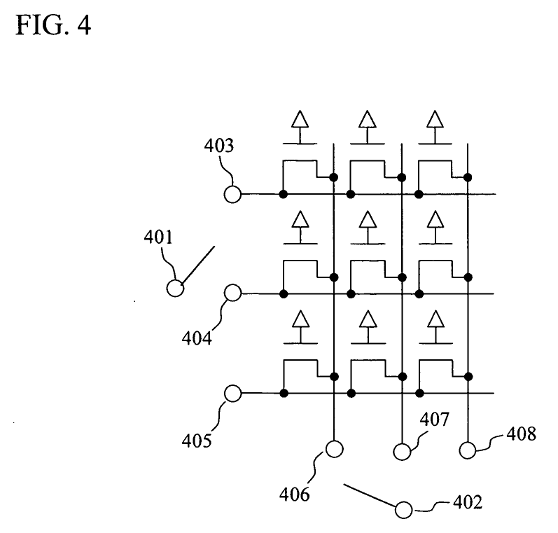

Also, a method for measuring the drain current from multiple FETs is implemented by a combination of connections of a source wiring switch 401 linked to source wiring of the signal processing circuit and a drain wiring switch 402 linked to drain wiring thereof to any one of sources 403, 404 and 405 of each FET and any one of drains 406, 407 and 408 thereof, respectively, as shown in FIG. 4.

To induce the extension reaction, the dATP solution feed valve 225 was opened (at step S308), a reaction solution containing KCl (50 mM), Tris-HCl (20 mM, pH 8.4), MgCl₂ (3 mM), Klenow Fragment (0.1 UμL⁻¹), and dATP (5 mM) in the dATP solution container 220 was developed into the solution tank 214 through the solution injection port 217, and the dATP solution feed valve 225 was closed (at step S309). The solution was caused to put at a temperature of 25 degrees for 3 minutes and thereby to react. After that, unreacted DATP that can possibly cause noise during the detection of the change in the interfacial potential was washed away, and further, the cleaning solvent feed valve 224 was opened (at step S310) in order to replace the solution by a low ion solution for the detection of the interfacial potential, the phosphate buffer solution (0.025 M Na₂HPO₃/0.025 M KH₂PO₃, pH 6.86) in the cleaning solvent container 219 was developed into the solution tank 214 through the solution injection port 217, and the cleaning solvent feed valve 224 was closed (at step S311).

In the same manner as mentioned above, a pressure wave was caused to generate (at step S312), the drain current was measured (at step S313), the average V₁ and standard deviation ΔV₁ of the interfacial potential change in each well after the extension reaction were obtained. As for each of the wells, it was determined that the extension reaction occurred when V₁−V₀>3×ΔV₀. After that, the cleaning solvent feed valve 224 was opened (at step S314), the phosphate buffer solution (0.025 M Na₂HPO₄/0.025 M KH₂PO₄, pH 6.86) in the cleaning solvent container 219 was developed into the solution tank 214 through the solution injection port 217, and the cleaning solvent feed valve 224 was closed (at step S315). The above steps were repeated multiple times in turn from dATP (at step S320) to dCTP (at step S317), dGTP (at step S318), and dTTP (at step S319) to thereby determine the target DNA sequence of the DNA molecules 232, 243 and 251 immobilized on the fine particles 212, 242 and 250. Analysis using this configuration enables to determine DNA sequence 50 bases or more.

In order to perform the solution change in the gaps between the fine particles 212, 242 and 250 and the metal electrodes 209, 241 and 249, respectively, and to induce the extension reaction of the target DNA present in the fine particles in the gaps therebetween, it is more effective to have some distance in each gap. In the first embodiment, at the time of the solution change or the extension reaction, the fine particle control unit 229 did not generate the pressure wave, and the Brownian motion of the fine particles was utilized to provide the distance. Alternatively, to provide the distance, a pressure wave generator may be installed under the FET sensor unit 233 shown in FIG. 2 as in the case of the fine particle control unit 229 so that the pressure wave generator can generate the pressure wave at the time of the solution change or the extension reaction.

Incidentally, in the first embodiment, the Klenow Fragment was used as DNA polymerase, and thus, the reaction was caused to occur at room temperature. However, if heat-resistant DNA polymerase such as Taq polymerase is used, it is desirable to add a mechanism for keeping the temperature in the chamber unit high.

In the first embodiment, DNA was used as a target for sequence decoding. However, RNA may also be used for purposes of gene expression analysis or the like. If RNA is used, RNA sequence can be determined by forming sampled RNA into a DNA chain by reverse transcription according to an existing method, and performing the subsequent process as in the case of the DNA described above.

Also, although emPCR was used as a means for immobilizing the same multiple target DNA molecules on the fine particles 212, 242 and 250, other means may also be used. For example, a method of pre-immobilizing single-stranded probe DNA molecules on the fine particles 212, 242 and 250 may be used as given below. The same multiple fragments having different sequence portions on a reference genome are immobilized as the probe DNA on the fine particles 212, 242 and 250, and the fine particles 212, 242 and 250 are disposed in the wells in the same manner as the above. A solution containing multiple single-stranded target DNA fragments is developed by the same solution feed means as the above to thereby effect hybridization. The target DNA fragments are ideally hybridized only with the probe DNA having a complementary sequence, and thus, the target DNA fragments are immobilized only on the given fine particles 212, 242 and 250. Thereafter, the DNA sequence determination method is equivalent to the above. The advantages of the method of immobilizing the single-stranded probe DNA are that (1) this method saves the trouble of removing the cover 216 for the sequence analysis process, because the FET sensor unit 233 and the chamber unit 234 having the fine particles 212, 242 and 250 immobilized thereon in the wells can be prepared beforehand; (2) this method enables efficient mapping because it is able to control the starting point of a sequence to be read, using a known sequence on the reference genome as the probe DNA, and so on.

### Second Embodiment

In the second embodiment, a magnetic field generator was used as a means for controlling the position of the fine particle. The general configuration of the system is equivalent to that shown in FIG. 2. In the second embodiment, a strong magnet was used as the magnetic field generator for the fine particle control unit 229, and magnetic beads of 200 μm in diameter were used for the fine particles 212, 242 and 250. A preparation method for the fine particles 212, 242 and 250 having the DNA molecules 232, 243 and 251 immobilized thereon, a method for settlement of the fine particles 212, 242 and 250 in the wells, and an extension reaction method are equivalent to those of the first embodiment. Description will be given below with regard to a solution change and a interfacial potential detection method, which are different from the first embodiment.

At the time of the solution change using the solution injection port 217 and the solution ejection port 218, for purposes of more efficient solution change, the magnet of the fine particle control unit 229 was disposed on the cover 216 (as shown in FIG. 2), and the fine particles 212, 242 and 250 were caused to move toward the cover 216.

For the interfacial potential detection, the magnet of the fine particle control unit 229 was disposed under the silicon substrate 204 shown in FIG. 2 so that the fine particles 212, 242 and 250 (i.e., the magnetic beads) would be pressed against the spherical surfaces of the metal electrodes 209, 241 and 249. As in the case of the first embodiment, the fine particles 212, 242 and 250 were pressed against the spherical surfaces, the interfacial potential change V₀ before the extension reaction and the interfacial potential change V₁ after the extension reaction were obtained, and it was determined whether or not the extension reaction occurred from a difference between the interfacial potential change V₀ and the interfacial potential change V₁. The above steps were repeated in turn from A to C, G, and T to thereby determine the target DNA sequence of the DNA molecules 232, 243 and 251 immobilized respectively on the fine particles 212, 242 and 250.

### Third Embodiment

Description will be given with reference to FIG. 2, as in the case of the first and second embodiments. In the third embodiment, temperature control is used to control the position of the fine particle for nucleotide sequence determination. To this end, a Peltier element for the temperature control is disposed in the fine particle control unit 229. Other structural components are equivalent to those of the first embodiment. Incidentally, in the third embodiment, the Peltier element is used; however, anything other than the Peltier element may also be used, as long as it is a system for temperature control. Also, a specific sequence determination method is equivalent to the first embodiment, except for a fine particle manipulation method for the interfacial potential detection method.

For the interfacial potential detection, the Peltier element of the fine particle control unit 229 was used to set the temperature in the solution tank 214 at 5 degrees. This suppresses the Brownian motion of the fine particles 212, 242 and 250, thus increasing the probability of the fine particles being in contact with the spherical surfaces of the metal electrodes 209, 241 and 249. The interfacial potential at this time was detected and set at V₀. For the extension reaction, the temperature in the solution tank 214 was reset at any one of 25 and 37 degrees. After the extension reaction, the temperature in the solution tank 214 was likewise set at 5 degrees, and the interfacial potential V₁ was obtained. It was determined whether or not the extension reaction occurred from a change in V₁ with respect to V₀. The above steps were repeated in turn from A to C, G, and T to thereby determine the target DNA sequence of the DNA molecules 232, 243 and 251 immobilized on the fine particles 212, 242 and 250. In the third embodiment, the temperature at the time of the interfacial potential detection was set at 5 degrees. However, the set temperature is not particularly limited, as long as it is more than 0 degree at which solution in the solution tank 214 freezes. However, it is required to reproducibly keep the same set temperature at the time of sequence determination, since the change in the interfacial potential before and after the extension reaction is to be compared.

### Fourth Embodiment

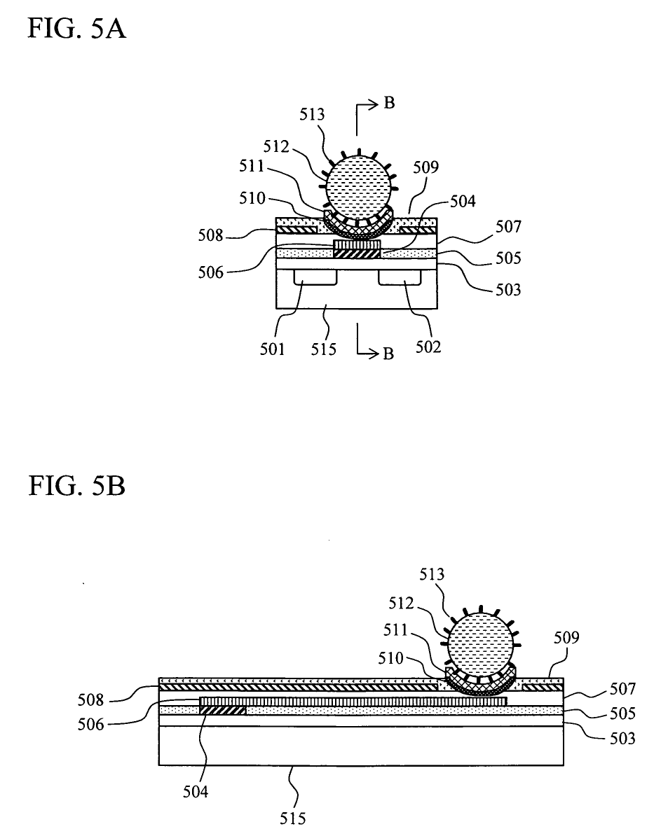

The fourth embodiment uses a FET sensor unit in which a conductive wiring 504 and a metal electrode 511 are spatially separated as shown in FIGS. 5A and 5B. FIG. 5A is a front view of the FET sensor unit, and FIG. 5B is a cross-sectional view taken along the line B-B of FIG. 5A. The conductive wiring 504 and the metal electrode 511 are spatially separated, thereby easily facilitating light shielding for reducing the entry of light, which can possibly cause noise, into the conductive wiring 504 and the gate. Description will be given below with regard to a fabrication method of the FET sensor unit for implementing the above configuration.

The chamber unit and the fine particle control unit 229 other than the FET sensor unit are equivalent to those of the first embodiment shown in FIG. 2. Also, a series of steps required for sequence determination is equivalent to those of the first embodiment. In the fourth embodiment, the configuration and operation other than the FET sensor unit followed the first embodiment. However, any one of the second and third embodiments may also be used.

Description will be given below with reference to FIGS. 5A and 5B with regard to the FET sensor unit that is different from the first embodiment. A source 501, a drain 502, a gate insulating film 503, and a conductive wiring 504, which form the basic configuration of the FET sensor, were fabricated in an array by an equivalent method to the first embodiment. Materials therefor were also equivalent to those of the first embodiment. Besides that, each of an insulating film 505, an extension wiring 506, an insulating film 507, a light shield film 508 and an insulating film 509 was also fabricated by performing coating of the materials, resist coating, patterning, development, and etching in this order as in the case of the first embodiment, thereby yielding the configuration shown in FIGS. 5A and 5B, except for an adhesive layer 510, a metal electrode 511 and a fine particle 512.

For fabrication of the metal electrode 511 having a spherical surface, dot patterning and development were performed on the insulating film 509 in a location where the metal electrode was to be disposed, and the patterned part was subjected to isotropic etching to form the spherical surface. After that, tungsten (W) was sputtered as the adhesive layer 510 for providing an electrical connection between the metal electrode 511 and the light shield film 508, and gold (Au) was sputtered as the metal electrode 511, and the metal electrode 511 having the spherical surface was formed in an array by resist coating, patterning, development, and etching in the same manner as described above. In the fourth embodiment, aluminum (Al) was used as the material for the light shield film 508. However, the material is not particularly limited, as long as it is capable of light shielding. Also, the material for the insulating films 505, 507 and 509 has to be an insulating material such as oxide. Although aluminum (Al) having lower resistance was used as the material for the extension wiring 506, polysilicon may also be used as in the case of the conductive wiring 504. The advantage of this is that the process for the conductive wiring 504 and that for the extension wiring 506 can be done at a time. Besides that, it is preferable to use a material having low resistance and good processability for etching or the like for the extension wiring 506, and molybdenum (Mo) or the like may be used. In the fourth embodiment, the light shield film 508 is formed in the FET sensor unit as mentioned above, thereby enabling the omission of the light shield operation for the interfacial potential measurement, as in the case of the first embodiment.

### EXPLANATION OF REFERENCE NUMERALS

101, 212, 242, 250 and 512 . . . fine particles
103, 209, 241, 249 and 511 . . . metal electrodes
201 . . . measuring unit
202 . . . signal processing circuit
203 . . . data processing device
204 and 515 . . . silicon substrates
205, 236, 245, 501, 403, 404 and 405 . . . sources
206, 237, 246, 502, 406, 407 and 408 . . . drains
207 and 503 . . . gate insulating films
208, 238, 247 and 504 . . . conductive wirings
210, 505, 507 and 509 . . . insulating films
211, 240, 248 and 510 . . . adhesive layers
213 and 244 . . . sectioning layers
214 . . . solution tank
215 and 252 . . . spacers
216 . . . cover
217 . . . solution injection port
218 . . . solution ejection port
219 . . . cleaning solvent container
220 . . . dATP solution container
221 . . . dTTP solution container
222 . . . dGTP solution container
223 . . . dCTP solution container
224 . . . cleaning solvent feed valve
225 . . . dATP solution feed valve
226 . . . dTTP solution feed valve
227 . . . dGTP solution feed valve
228 . . . dCTP solution feed valve
229 . . . fine particle control unit
230 . . . reference electrode
231 . . . high-frequency power supply
232, 243, 251 and 513 . . . DNA molecules
233 . . . FET sensor unit
234 . . . chamber unit
235 . . . reference electrode unit
401 . . . source wiring switch
402 . . . drain wiring switch
506 . . . extension wiring
508 . . . light shield film

What is claimed is:

1. A DNA measuring system, comprising:
a container that accommodates a measuring solution containing DNA polymerase;
a spherical support having any one of single-stranded DNA and double-stranded DNA bonded to the surface, the support being placed in the measuring solution;
a sensor having an electrode having formed on the surface a spherical recess fitted to the shape of the support, the sensor being configured to detect an electrical state in a region in the vicinity of the electrode; and
a means for selectively injecting a solution containing any one of dNTP (where N denotes any one of A, C, G and T) and a derivative thereof,
wherein the support is brought into close contact with the surface of the electrode for detection of the electrical state.

2. The DNA measuring system according to claim 1, further comprising a mechanism for controlling the relative position of the support with respect to the electrode.

3. The DNA measuring system according to claim 2, wherein the mechanism has a pressure wave generating function.

4. The DNA measuring system according to claim 2, wherein the mechanism has a magnetic field generating function, and the support is a magnetic material.

5. The DNA measuring system according to claim 2, wherein the mechanism has a temperature control function.

6. The DNA measuring system according to claim 1, wherein the following relationship is satisfied:
r₂−\sqrt{r₂²−2(1−cos θ)r₁r₂+2(1−cos θ)r₁²}<D
r₁<r₂
2π(1−cos θ)r₁²d>IᵣtₐC
where r₁ denotes the radius of the support; r₂, the curvature radius of the recess on the surface of the electrode; θ, a parameter that gives a solid angle 2π(1−cos θ) in a direction of a contact point between the support and the recess from the center of the support when the support is in contact with the recess on the surface of the electrode; D, Debye length; d, the density of the DNA immobilized on the support; Iᵣ, the leakage current value of the sensor; tₐ, the time interval between detections of the electrical state; and C, a coefficient of conversion from coulomb to electron number.

7. The DNA measuring system according to claim 1, wherein the sensor includes an insulated gate field effect transistor, and the electrode is connected to a gate of the insulated gate field effect transistor by a conductive wiring.

8. The DNA measuring system according to claim 1, wherein the electrode is made of a noble metal.

9. The DNA measuring system according to claim 1, wherein a plurality of the sensors are disposed on one and the same substrate.

10. The DNA measuring system according to claim 9, wherein a sectioning layer that sections a plurality of wells is included in the container, and the sensor and the support, one each, are placed in each of the wells.

11. The DNA measuring system according to claim 10, wherein the container has a lid, and a height from a top surface of the sectioning layer to the lid is smaller than the diameter of the support.

12. The DNA measuring system according to claim 7, wherein the electrode is located on a substrate at a position spatially separated from immediately above the gate.

13. The DNA measuring system according to claim 12, wherein the gate is coated with a light shield film.

14. A DNA measuring method, comprising the steps of:
bonding a target DNA to the surface of a spherical support having a single-stranded DNA immobilized thereon;
immersing the support having the target DNA bonded thereto into a solution containing polymerase;
injecting selectively a solution containing any one of dNTP (where N denotes any one of A, C, G and T) and a derivative thereof;
moving the support toward the surface of an electrode of a sensor, the sensor having the electrode having formed on the surface a spherical recess fitted to the shape of the support, the sensor being configured to detect an electrical state in a region in the vicinity of the electrode; and
measuring the electrical state in the region in the vicinity of the electrode by the sensor.

15. The DNA measuring method according to claim 14, wherein the moving step is synchronized with the measuring step.

16. The DNA measuring method according to claim 14, further comprising the steps of:
changing the solution; and
moving the support away from the surface of the electrode,
wherein the two steps are synchronized.

17. The DNA measuring method according to claim 14, wherein a pressure wave is used for the moving step.

18. The DNA measuring method according to claim 14, wherein a magnetic field is used for the moving step.

19. The DNA measuring method according to claim 14, wherein a change in temperature is used for the moving step.

20. The DNA measuring method according to claim 16, wherein one and the same mechanism is used for the step of moving the support toward the surface of the electrode and the step of moving the support away from the surface of the electrode.
